(19) United States
(12) Patent Application Publication
(10) Pub. No.: US 2005/0288691 A1
(43) Pub. Date: Dec. 29, 2005

(54) Hernia patch

(76) Inventor: Arnold R. Leiboff, Stony Brook, NY (US)

Correspondence Address: BRIAN ROFFE, ESQ, 11 SUNRISE PLAZA, SUITE 303, VALLEY STREAM, NY, 11580-6170, US

(21) Appl. No.: 10/878,102

(22) Filed: Jun. 28, 2004

(51) Int. Cl. A61B 17/08

(52) U.S. Cl. 606/151

(57) ABSTRACT

A surgical prosthesis including a sheet formed of one or more layers of a thin flexible material suitable to promote or prevent biological tissue adherence thereto and a grid formed of intersecting lines provided on at least one main surface of the sheet. The grid helps the surgeon trim the prosthesis to proper size, deploy the prostheses into optimal position and fix the prosthesis with optimal alignment and tension. The prosthesis may be used as a hernia patch.

### FIELD OF THE INVENTION

The present invention relates generally to a surgical prosthesis for hernia repair and, more particularly, to a surgical prosthesis for hernia repair provided with a grid on at least one main surface to ensure correct positioning of the prosthesis and optimal placement of fasteners (sutures, tacks, staples or clips) used to secure the prosthesis during hernia repair. The present invention also relates to a method for making the surgical prosthesis.

### BACKGROUND OF THE INVENTION

Surgical prostheses of various different types have long been used for hernia repair. An abdominal wall hernia develops when structures normally contained by the abdominal wall penetrate (herniate) into or through the abdominal wall through a defect in the fascial layer, the strong layer that normally maintains the integrity of the abdominal wall. In modern practice, hernia repair is usually performed by installing a surgical prosthesis, commonly known as a hernia patch (or mesh), over a fascial defect. The patch is fixed to the surrounding tissue with fasteners. The hernia patch prevents the herniation of the abdominal viscera through a defect in the fascial layer. This technique is preferred over direct suture closure of the fascial defect because it avoids placing excessive tension on the musculofascial tissue and thereby makes it less likely for the hernia to recur. Hernia repair with prosthetic patches can be accomplished via an open or laparoscopic approach.

There are several types of prosthetic materials used in hernia repair. One example is polypropylene mesh, which is commercially available under the registered trademarks MARLEX and SURGIPRO. Another example is expanded polytetrafluoroethylene (ePTFE) which is commercially available under the registered trademark GORTEX. These and other conventional prosthetic materials are widely used in hernia repair and are disclosed, for example, in U.S. Pat. Nos. 5,368,602; 5,356,432; 5,326,355; 5,292,328; 5,290,217; 5,254,133; and 5,147,374.

Hernia patches are marketed in various sizes and shapes. But hernia defects vary greatly in configuration. Therefore, the prosthesis often needs to be trimmed to optimal size and shape. When necessary, this is usually done freehand by the surgeon, which sometimes produces a patch with less than ideal configuration.

Hernia patches are formed of a thin, flexible material. As a result, a great deal of care must be exercised by the surgeon when installing a hernia patch. The limited visibility and maneuverability available to the surgeon, and the fact that many hernia patches tend to become folded, stretched or gathered when being positioned, may lead to disorientation and improper placement and/or fixation of a hernia patch. This may result in a failure to cover the entire hernia defect, or improper tension on the patch. Such errors may result in the recurrence of the hernia.

The foregoing problems may be overcome by providing a hernia patch with a guide or template that would be effective in assisting the surgeon in trimming and installing the hernia patch. It would be most desirable to provide a hernia patch with one or more types of indicia that would help the surgeon ensure that the hernia patch has been properly sized, shaped and positioned and would provide the surgeon with visual guides for fixing the hernia patch to the tissue surrounding the fascial defect. One example of a prosthesis which provides visual guides is the Ethicon-EndoSurgery SOFT PROLENE® Mesh, which has horizontal blue stripes which help orient the mesh when deployed. Two products from W. L. Gore & Associates, Inc., Dualmesh® Emerge and Dualmesh® Emerge Plus, provide a Control Layer with elastic memory which prevents distortion of the attached biomaterial to facilitate placement (see FIG. 1). This Control Layer has visual guides which aid suture and tack placement. When the biomaterial is affixed to the abdominal wall, the EMERGE Control Layer is removed, leaving only the supple biomaterial in place.

### OBJECTS AND SUMMARY OF THE INVENTION

An object of the present invention is to provide a surgical prosthesis, which can be more efficiently and accurately trimmed to size and shape and installed, resulting in shorter operative times, diminished patient morbidity and fewer hernia recurrences.

Another object of the present invention is to provide a new method for forming a surgical prothesis.

In accordance with the present invention, one or both main surfaces of the flexible sheet prosthesis are provided with a grid of indices which helps the surgeon trim the prosthesis to proper size, deploy the prostheses into optimal position, and fix the prosthesis with optimal alignment and tension.

An example of a surgical procedure utilizing the inventive surgical mesh prosthesis is disclosed herein, but is not intended to limit the present invention to a single type of surgery. For example, the surgical prosthesis of the present invention may be utilized in a ventral hernia operation wherein the inventive surgical mesh prosthesis invention is used to repair a defect in the anterior abdominal wall. However, the inventive prosthesis may also be used in the surgical treatment of other types of hernias as well as in thoracoplasty, or the like.

The foregoing and other advantages of the present invention will become clear in the following detailed description of the preferred embodiments taken together with the attached drawings, wherein like reference numerals represent like elements throughout.

### BRIEF DESCRIPTION OF THE DRAWINGS

FIG. 1 is a view of a prior art device showing the EMERGE Control Layer, with its visual fixation guides, on DUALMESH® Biomaterial;

FIG. 2A is a perspective view of a circular surgical mesh prosthesis according to the present invention;

FIG. 2B is a perspective view of a circular surgical mesh prosthesis with indices along each radial marking;

FIG. 2C is a perspective view of a circular surgical mesh prosthesis with concentric circular markings in addition to radial markings;

FIG. 3A is a perspective view of an elliptical surgical mesh prosthesis according to the invention;

FIG. 3B is a perspective view of an elliptical surgical mesh prosthesis with indices marked along each radial marking;

FIG. 3C is a perspective view of an elliptical surgical mesh prosthesis with concentric elliptical markings in addition to radial markings;

FIG. 4A is a perspective view of a rectangular surgical mesh prosthesis according to the invention;

FIG. 4B is a perspective view of a rectangular surgical mesh prosthesis with indices marked along each radial marking;

FIG. 4C is a perspective view of a rectangular surgical mesh prosthesis with concentric obround markings in addition to radial markings;

FIG. 5A is a perspective view of a composite two layer prosthesis with grid marks seen on its upper surface;

FIG. 5B is a cross-sectional view of a composite two layer prosthesis;

FIG. 6A is a cross-sectional view illustrating an operative position of the surgical mesh prosthesis illustrated in FIG. 2A in a ventral hernia operation;

FIG. 6B is an elevated cross-sectional view illustrating an operative position of the surgical mesh prosthesis illustrated in FIG. 2A in a ventral hernia operation;

FIG. 6C is a top view illustrating an operative position of the surgical mesh prosthesis illustrated in FIG. 4A in a ventral hernia operation; and

FIGS. 7A and 7B are perspective views of additional embodiments of the inventive surgical prosthesis.

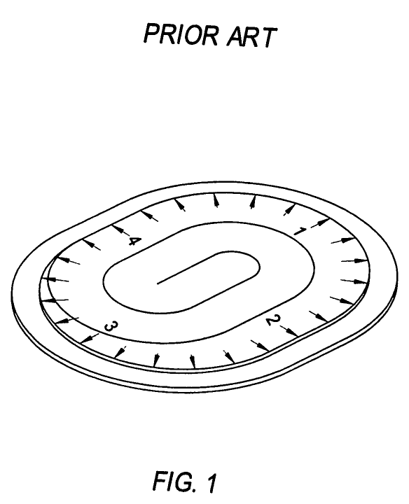

### DETAILED DESCRIPTION OF THE PREFERRED EMBODIMENTS

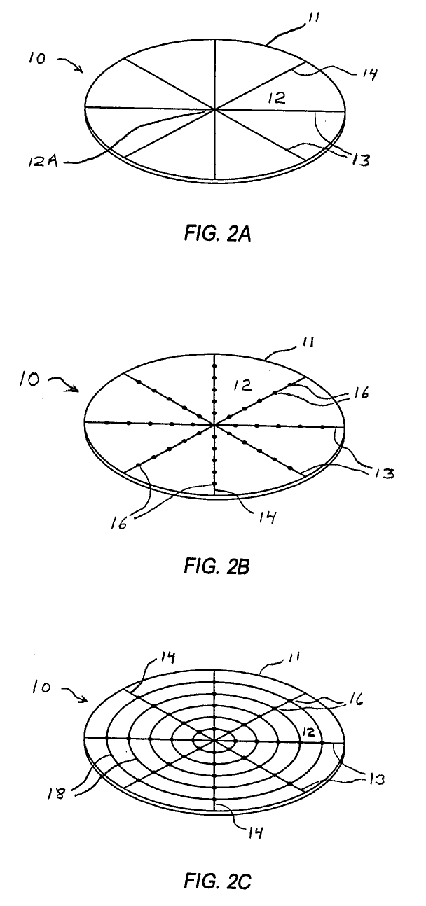

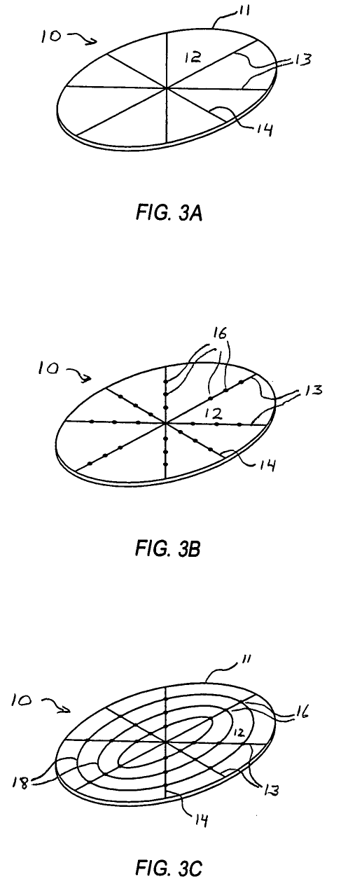

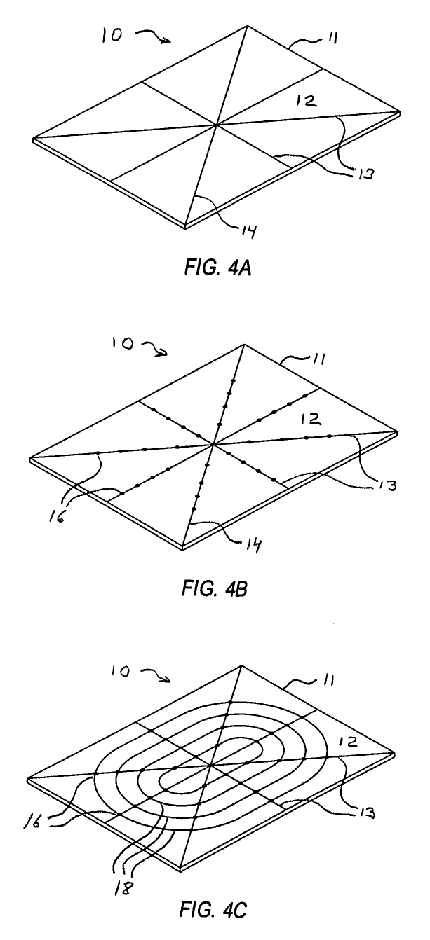

Referring to the accompanying drawings, FIG. 2A illustrates a surgical prosthesis 10 in the form of a flexible sheet 11 which is particularly well adapted for hernia repair. In the presently described embodiment, the inventive surgical prosthesis 10 is utilized as a hernia patch, although it is not limited to such use. Reference to element 10 as a hernia patch therefore does not limit the use of the prosthesis in accordance with the invention. The hernia patch 10 can be formed as a circular sheet 11, as shown in FIG. 2A in a range of sizes. Alternatively, the hernia patch 10 may be preformed in the shape of an ellipse (FIGS. 3A-3C), rectangle (FIGS. 4A-4C) or any particular shape desired.

As can also be seen in FIG. 2A, at least one main surface 12 of the hernia patch 10 is provided with a grid 13. In the preferred embodiment, the grid 13 comprises a series of radial lines 14 that intersect at a central point 12A. The grid 13 may be imprinted directly onto the hernia patch 10 or may be formed of any suitable means, such as stitching having a different color from that of the surrounding material of the hernia patch 10.

The grid 13 provides the surgeon with visual guides to help trim the patch 10 to the desired shape and size, position the patch 10 relative to the fascial defect, and fix the patch 10 to the abdominal wall.

Defects in fascia may be circular, elliptical or other configuration. There may be solitary defects, or multiple. Fascial defects may be small or gigantic. Prostheses for hernia repair, however, are made available in limited shapes and sizes and often must be trimmed by the surgeon during the hernia operation to create a patch appropriate to the size and shape of the fascial defect, or, if multiple defects are present, to create a patch which can cover them all. The indices provided by the surgical prosthesis of this invention help the surgeon trim the patch to optimal configuration.

In the presently described embodiments, the grid 13 is formed of radial lines 14 that intersect at a central point 12a. Thus, symmetry of the hernia patch 10 can be achieved by trimming of the hernia patch 10 by the surgeon symmetrically about the central point 12A for circular patches, or around co-linear radial markings (i.e., a line marking a diameter) to produce a substantially elliptical patch. Additionally, indices 16 and 18 such as those illustrated in the embodiments illustrated in FIGS. 2B, 2C, 3B, 3C, 4B and 4C guide the surgeon as he trims the prosthesis, allowing him to trim the prosthesis faster and more accurately. Indices 16 are markings (e.g., short lines or dots) provided at equidistant points along each of the radial lines 14. Indices 18 are parallel markings that extend about all of the radial lines 14, forming concentric circles, ellipses, obrounds or the like, each equidistant from its neighbors.

It is important that the patch 10 be positioned properly relative to the fascial defect. An overlap where the patch 10 extends beyond the edge of the fascial defect for two to five centimeters is known to be optimal. The markings 14, 16, 18 on the surgical prosthesis of this invention help the surgeon to orient the patch 10 so that it is optimally positioned relative to the fascial defect, so that amount of overlap is optimal along the entire circumference of the fascial defect. For example, if the fascial defect is circular, and the prosthesis is properly trimmed, the point where all radial lines intersect should be situated in the center of the fascial defect.

Finally, correct placement of the fasteners (sutures, tacks, staples or clips) into the prosthesis is often problematic, because of the tendency of some prosthetic materials, such as expanded PTFE, which is supple and slightly elastic, to fold, crimp, stretch, gather, and otherwise deform. The surgeon can easily lose his orientation and, as a result, misplace fasteners so that overlap between the patch and fascial defect is too small in some areas, too large in others, and/or the patch is not under proper tension. Where the flap is too tight, it may tear away from the abdominal wall. If too loose, the abdominal viscera may protrude into or through the plane of the abdominal wall, producing a cosmetically and functionally undesirable bulge. The grid provides the surgeon with a guide or template that facilitates the fixation of the hernia patch to the abdominal wall. During a surgical procedure, the surgeon may apply a suture along a line in the grid, and use that suture to secure the mesh at a corresponding point on the abdominal wall. The radial markings allow him to know exactly where to place his next stitch along the circumference of the patch, even though the patch may have been temporarily stretched or otherwise deformed.

As mentioned, it is often necessary to trim the patch. Even when the inventive surgical prosthesis is trimmed by the surgeon, the radial markings on the resulting hernia patch still provide the surgeon with valid visual guides to insure proper placement of the patch and proper spacing and distribution of the fasteners.

The hernia patch 10 may be formed of a single layer of a material that fosters biological tissue adherence thereto. These materials include polyolefins such as polypropylene or polyethylene. Alternatively, the hernia patch 10 may be formed of a layer of a material that impedes biological tissue adherence thereto. These materials include fluoropolymers such as expanded polytetrafluoroethylene (ePTFE), fluorinated ethylene propylene (FEP), tetrafluoroethylene (TFE) and ethylene tetrafluoroethylene (ETFE).

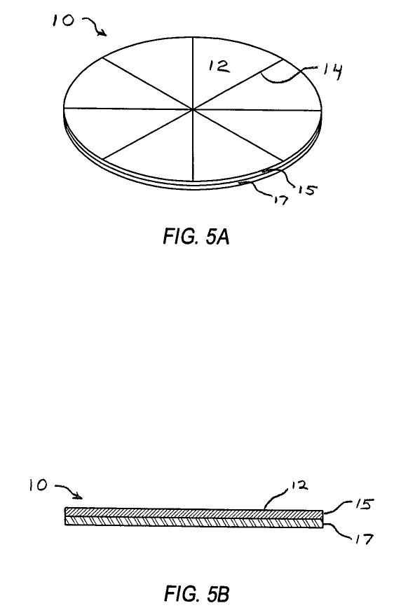

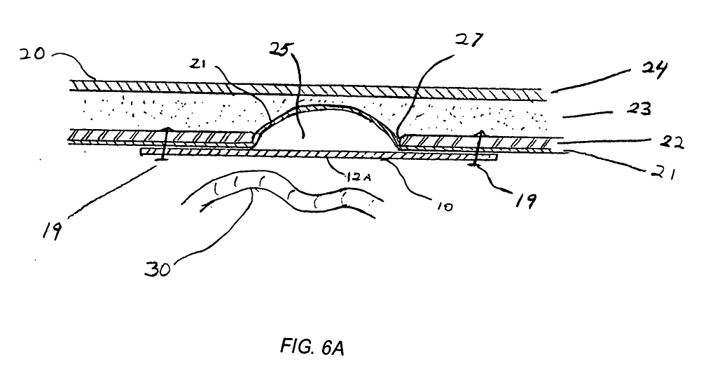

As will be appreciated by those of ordinary skill in the art, the hernia patch 10 may also have a two-layer structure 15, 17 (see FIGS. 5A and 5B). One layer 15 may be formed of a material that fosters tissue ingrowth and adherence and the second layer 17 of a material that impedes tissue ingrowth and adherence. If the hernia patch 10 is placed within the peritoneal cavity against the anterior abdominal wall, the side of the combination hernia patch which fosters tissue ingrowth is placed against the anterior abdominal wall, while the side which impedes tissue ingrowth faces the abdominal viscera. Thus, the patch 10 becomes well incorporated and securely attached to the abdominal wall, yet does not form undesirable and potentially harmful adhesions with the viscera. Grid lines 14, as described by this invention may be placed on either or both surfaces of a two-layer patch (see FIG. 5A), providing the surgeon with the same advantages provided in the one-layer hernia patch.

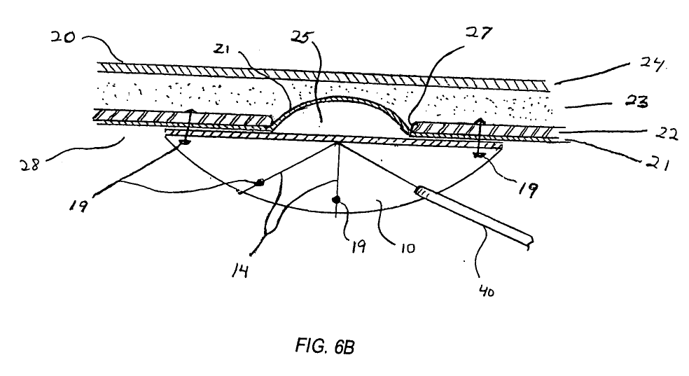

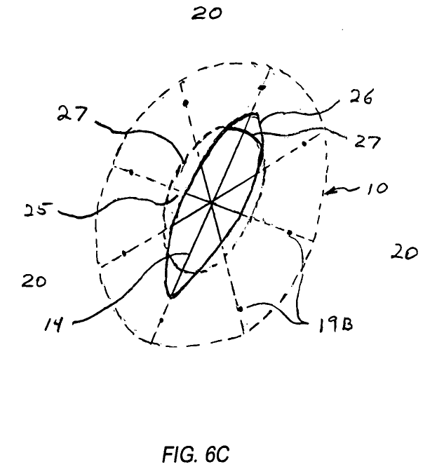

Hernia operations to illustrate the advantages of the prosthesis of this invention are now described with reference to FIGS. 6A-6C.

FIG. 6A is a cross-sectional view illustrating a hernia patch 10 formed from a surgical prosthesis of this invention in a ventral hernia operation. In such an operation, the hernia patch 10 is positioned over the defect 25 in the fascial layer 22 so that it extends beyond the edge 27 of the fascial defect. The point 12A where the radial markings (not seen in the cross-sectional view 6A) intersect is placed at the center of the fascial defect 25. The patch 10 is fixed to the peritoneal surface of the anterior abdominal wall 20 by tacks 19. For clarity, various layers of the abdominal wall are shown, including the peritoneal lining 21 of the abdominal cavity, the fascia 22, subcutaneous layer 23 and skin 24. Also the viscera are represented by a segment of intestine 30, which may come into contact with the prosthesis.

FIG. 6B shows a cross-sectional elevation view of the hernia patch and anterior abdominal. Here the surgeon looks up at the hernia patch 10 and the posterior surface 28 of the anterior abdominal wall 20 from within the abdominal cavity, as he would do in a laparoscopic repair of a ventral hernia. The hernia patch 10 has already been trimmed to proper size, inserted into the abdominal cavity through a port site, deployed over the hernia defect 25, and is now being tacked to the abdominal wall 20. Several tacks 19 have already secured the patch to the abdominal wall at multiple points evenly distributed along the periphery of the patch 10. The radial markings 14 on the posterior surface of the patch 10 serve as visual guides for the tacking. The end of a surgical tacking device 40 can be seen deploying another tack into the patch 10 and abdominal wall 20. The tacks 19 can be seen to penetrate the fascial layer 22, which is necessary for secure fixation.

FIG. 6C shows a top view of an incision 26 in the anterior abdominal wall 20, created for open ventral hernia repair. The hernia patch 10 has already been trimmed to proper size and elliptical shape, inserted into the abdominal cavity through the incision 26 and deployed to cover the hernia defect 25. Within the incision the radial markings 14 of the elliptical hernia patch 10 can be directly seen. Those portions of the radial markings which extend outside the edges of the incision and the border of the hernia patch are represented by hatched lines because they are out of view, being situated behind the anterior abdominal wall. Through the incision 26, the surgeon is able to place his sutures 19B to secure the periphery of the patch 10 to the anterior abdominal wall 20. As mentioned, these sutures 19B (or tacks or other fasteners) should include the fascial layer 22 for proper fixation of the patch 10. The radial markings 14 serve as visual guides for the surgeon to optimally position and space the sutures 19B.

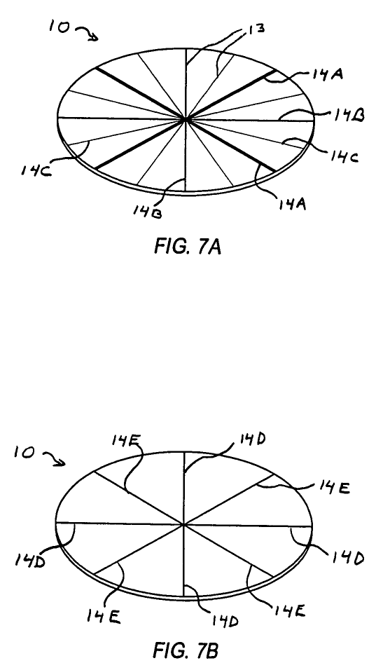

FIGS. 7A and 7B illustrate additional embodiments of the hernia patches 10 of the present invention. FIG. 7A shows the grid 13 to include thick radial lines 14A, lines of medium thickness 14B, and thin radial lines 14C. The width of the lines serve to inform the surgeon which points on the circumference are diametrically opposed to which other points, which further guides the surgeon in positioning and fixing the patch. A similar objective can be achieved by using different colors to distinguish among the radial markings. In FIG. 7B, blue radial markings 14D alternate with red radial markings 14E.

The hernia operations described above are merely intended to be two examples of the type of use of the inventive hernia patch. As will be readily appreciated by those of ordinary skill in the art, a large number of hernia operations can be performed utilizing the inventive patch.

The patch 10 of the present invention, while particularly well suited for hernia operations, is not specifically limited thereto and may have various other applications such as thoracoplasty or in vascular surgery. For example, the hernia patch 10 shown in FIG. 2A can be used for the reconstruction of the thoracic wall. In vascular surgery, the patch 10 may be preformed in a tubal fashion where the outer surface of the tube is provided with a grid of parallel lines.

Although the invention has been described with particularity, it will be apparent to those of ordinary skill in the art that various modifications and changes may be made thereto without departing from the spirit and scope thereof. Consequently, the scope of the present invention is intended to be defined by the attached claims.

1. A hernia patch comprising:
a sheet formed of one or more layers of a thin flexible material suitable to promote or prevent biological tissue adherence thereto; and
a grid formed of intersecting lines provided on at least one main surface of the sheet.

2. A hernia patch according to claim 1, wherein the grid comprises a plurality of lines intersecting at a central point.

3. A hernia patch according to claim 2, further comprising indices at equidistant points along each of the lines.

4. A hernia patch according to claim 2, further comprising indices that extend about all of the lines.

5. A hernia patch according to claim 1, wherein the grid comprises a plurality of straight lines extending between opposite edges of said sheet and intersecting at a common point.

6. A hernia patch according to claim 1, wherein the sheet comprises a first layer formed of a material that promote biological tissue adherence thereto and a second layer provided on the first layer and formed of a material that prevents biological tissue adherence thereto.

7. A hernia patch according to claim 1, wherein the sheet is formed of a layer of polypropylene.

8. A hernia patch according to claim 1, wherein the sheet is formed of a layer of expanded PTFE.

9. A hernia patch according to claim 1, wherein the sheet is formed of a layer of a fluoropolymer.

10. A hernia patch according to claim 1, wherein the lines are imprinted on the sheet.

11. A hernia patch according to claim 1, wherein the lines are stitches provided in the sheet and having a different color from that of the sheet.

12. A hernia patch according to claim 1, wherein the lines have varying thicknesses.

13. A hernia patch according to claim 1, wherein the sheet is one of circular, elliptical, rectangular, square or obround.

14. A method for making a hernia patch, comprising the steps of:
forming a sheet of one or more layers of a thin flexible material suitable to promote or prevent biological tissue adherence thereto; and
forming a grid of intersecting lines on at least one main surface of the sheet.

15. A method according to claim 14, wherein the grid is formed with a plurality of lines intersecting at a central point.

16. A method according to claim 15, further comprising the step of forming indices at equidistant points along each of the lines.

17. A method according to claim 15, further comprising the step of forming indices that extend about all of the lines.

18. A method according to claim 14, wherein the step of forming the grid comprises the step of imprinting the grid on the at least one main surface of the sheet.

19. A method according to claim 14, wherein the step of forming the grid comprises the step of arranging stitches on the at least one main surface of the sheet, the stitches having a different color from that of the sheet.
